(19) United States
(12) Patent Application Publication
(10) Pub. No.: US 2022/0233472 A1
(43) Pub. Date: Jul. 28, 2022

(54) VETERINARY BROMHEXINE HYDROCHLORIDE SOLUBLE POWDER, PREPARATION METHOD AND USE THEREOF

(71) Applicant: Qingdao Agricultural University, Qingdao City (CN)

(72) Inventor: Yongda ZHAO, Qingdao City (CN)

(73) Assignee: Qingdao Agricultural University, Qingdao City (CN)

(21) Appl. No.: 17/576,943

(22) Filed: Jan. 15, 2022

(30) Foreign Application Priority Data
Jan. 22, 2021 (CN) 202110090479.2

(51) Int. Cl. A61K 31/137; A61K 47/12; A61K 9/16; A61K 47/26

(52) U.S. Cl. CPC A61K 31/137; A61K 47/26; A61K 9/1694; A61K 47/12

(57) ABSTRACT

A veterinary bromhexine hydrochloride soluble powder, a preparation method and use thereof are disclosed. The method includes: (1) weighing a prescription amount of bromhexine hydrochloride and a part of prescription amount of citric acid, performing a primary mixing, pulverizing, sieving and dividing the mixed material into three equal parts; (2) weighing a remaining prescription amount of citric acid, pulverizing, sieving and dividing it into three equal parts; (3) weighing a prescription amount of lactose, sieving, and weighing three parts of the lactose accounting for 10% of the total amount; (4) putting one part of the mixed material obtained in “1”, one part of citric acid obtained in “2” and one part of lactose obtained in “3” alternately into a multi-directional motion mixer; (5) notifying an QA to inspect semi-finished products; and (6) packaging qualified semi-finished products, and warehousing qualified finished products.

### CROSS REFERENCE TO RELATED APPLICATIONS

This application claims the benefit and priority under 35 U.S.C. § 119(a) to Chinese patent application 202110090479.2 filed on Jan. 22, 2021, the entire teachings of which are incorporated herein by reference.

### BACKGROUND OF THE INVENTION

### Field of the Invention

The present invention relates to veterinary drugs, and in particular, to a veterinary bromhexine hydrochloride soluble powder, a preparation method and use thereof.

### Description of the Related Art

Bromhexine hydrochloride was developed by Boehringer Ingelheim, Germany, and was marketed in the 1960s, with the trade name of “Bisolvon”. It was also marketed in China in the 1970s. Boehringer Ingelheim has conducted systematic preclinical pharmacological, toxicological and clinical researches on bromhexine hydrochloride. Bromhexine hydrochloride was first marketed in Germany, and has now been marketed in most countries in the world, including China, and has become a best-selling expectorant drug.

Bromhexine hydrochloride, as a sputum dissolving agent, can directly act on bronchial glands to promote the release of lysosomes from mucus secreting cells, and differentiate and lyse the sticky sugar fibers in sputum, enabling sputum to be diluted and easy to cough up. In clinical practice, bromhexine hydrochloride is mainly used for acute and chronic bronchitis, asthma, bronchiectasis, and emphysema, and especially suitable for those having difficulty in coughing up white phlegm and critically ills caused by extensive sputum obstruction of small bronchial tubes. In human medicine, bromhexine hydrochloride formulations include oral tablets, oral solutions, aerosols, injections, freeze-dried powder injections, infusion solutions and other dosage forms. At present, it has been included in the pharmacopoeias of many countries such as China, Europe and Japan. In veterinary medicine in other countries, bromhexine hydrochloride is also approved for veterinary use. The target animals include pigs, cattle, dogs, chickens, etc. In China, there are no bromhexine hydrochloride-related formulations on the market currently.

At present, drugs for the treatment of respiratory diseases of livestock and poultry mainly target pathogenic microorganisms, and there are no specific drugs used in veterinary clinics for the clinical symptoms of livestock and poultry respiratory diseases, such as the inability to discharge sputum normally. Bromhexine hydrochloride has a significant protective effect on respiratory system. When being used in combination with antibiotics, bromhexine hydrochloride can increase the concentration of antibiotics in respiratory system and lesion locations, reduce the risk of infectious diseases, and have a certain synergistic effect, and meanwhile can decrease and break mucopolysaccharide fibers in sputum, thus reducing the viscosity of sputum and making sputum thinner and easier to cough up. Besides, bromhexine hydrochloride can inhibit the synthesis of acid glycoproteins in mucous membrane and goblet cells, reduce the content of sialic acid (one of the acidic mucopolysaccharide components) in sputum, and reduce the viscosity of sputum, thus being conducive to sputum expectoration. Therefore, the development of bromhexine hydrochloride-related formulations has important clinical significance for the treatment of respiratory diseases for livestock and poultry.

Currently, among the patents applied in China, one patent application No. 201610776470.6 discloses a bromhexine hydrochloride soluble powder and a preparation method and use thereof. In this patent, co-solvents, stabilizers and (E≤-cyclodextrin are used, and the method has a complicated preparation process, poor effect, and high cost, which greatly increases the breeding cost of the breeding industry, thereby being not conducive to industrialization and marketization.

Another patent application No. 202010801501.5 discloses a bromhexine hydrochloride soluble powder for poultry and a preparation method and use thereof. In this patent, co-solvents and soluble fillers are used. According to the dosage of co-solvents and the preparation method disclosed in this patent, the prepared bromine hydrochloride soluble powder takes a long time to dissolve and cannot be completely dissolved, and the drug will be further precipitated after standing.

Besides, based on the examples of this patent, it can be seen that the moisture content of the sample is only about 0.3%, while the moisture content of lactose is about 4.5-5.5%, the moisture content of citric acid is about 7.5-9.0%, and the proportion of lactose is as high as 90% or more. Therefore, the preparation process described in this patent is inconsistent with the data in the examples, indicating that the co-solvent and the preparation method in this patent cannot meet the needs of industrialization well.

Through the above analysis, it can be known that the problems and defects existing in the prior art are as follows:

(1) the developed formulations have more raw and auxiliary materials, leading to higher cost and increasing the burden of breeding for farmers;
(2) the amount of co-solvent used is insufficient, and the raw and auxiliary materials in the preparation process have relatively large pulverized particles, and thus the solubility problem cannot be completely solved; and
(3) in the used pulverization preparation process, the main component of the drug is mixed with the co-solvent in a ratio of 1:2, then the resulting mixture is directly mixed with 97% soluble auxiliary material, which cannot achieve a uniform content due to the low content (1%) of the main component.

The difficulty to solve the above problems and defects includes:

further reducing the application of auxiliary materials, production cost and the particle size of the raw and auxiliary materials, improving the preparation process, and increasing the uniformity of the content of the preparation to meet the requirements of Chinese Veterinary Pharmacopoeia on uniformity and solubility of low-content drugs.

It is significant to solve the above problems and defects. By solving the above problems and defects, the solubility of preparations and the stability of drugs in water could be improved, the breeding cost could be reduced, and thereby the social and economic benefits could be improved.

### BRIEF SUMMARY OF THE INVENTION

Aiming at the problems in the prior art, the present disclosure provides a veterinary bromhexine hydrochloride soluble powder, a preparation method and use thereof.

The present disclosure is realized by the following technical solutions:

An object of the present disclosure is to provide a veterinary bromhexine hydrochloride soluble powder, including 1-2% by mass of bromhexine hydrochloride, 10-20% by mass of a co-solvent (citric acid), with the rest being a soluble auxiliary material (lactose).

Another object of the present disclosure is to provide a method for preparing the veterinary bromhexine hydrochloride soluble powder. The method for preparing the veterinary bromhexine hydrochloride soluble powder includes:

step 1: accurately weighing a prescription amount of bromhexine hydrochloride and a part of prescription amount of citric acid, performing a primary mixing for a certain time or a manual primary mixing, putting the resulting mixed material after the primary mixing into a pulverizer and pulverizing, and sieving the mixed material after the pulverizing and dividing the mixed material into three equal parts for later use;

step 2: weighing a remaining prescription amount of citric acid, putting the citric acid into a pulverizer and pulverizing, and sieving the citric acid after the pulverizing and dividing the citric acid into three equal parts for later use;

step 3: weighing a prescription amount of lactose, sieving the lactose, and weighing three parts of the lactose accounting for 10% of the total amount of the lactose for later use;

step 4: putting one part of the mixed material obtained in step 1, one part of the citric acid obtained in step 2 and one part of the lactose obtained in step 3 alternately into a multi-directional motion mixer;

step 5: after mixing, notifying an on-site quality assurer (QA) to take a semi-finished product for inspection, and inspecting morphology, solubility, moisture, content and content uniformity; and

step 6: split charging, sealing and packaging the semi-finished product after being qualified through inspection, and warehousing a finished product after being qualified through inspection.

In some embodiments, in step 1, the citric acid is in an amount of 30% of the prescription amount, and the time for the primary mixing is 10 minutes.

In some embodiments, in step 1, the sieving is performed by a 100-mesh sieve.

In some embodiments, in step 2, the sieving is performed by a 100-mesh sieve.

In some embodiments, in step 3, the sieving is performed by an 80-mesh sieve.

In some embodiments, in step 4, putting the materials into the multi-directional motion mixer is performed as follows: after each one part of the material is added, the materials are mixed for 10 minutes; then the remaining amount of lactose is added at one time and the materials are mixed for 20 minutes; wherein the content uniformity is monitored depending on the condition of the mixing equipment.

Another object of the present disclosure is to provide a drug for auxiliary treatment of respiratory diseases of livestock and poultry, wherein the drug is prepared by using the veterinary bromhexine hydrochloride soluble powder.

By all the above technical solutions, the present disclosure has the following advantages and beneficial effects:

The present disclosure makes it possible to further reduce the particle size of the raw and auxiliary materials through an ultrafine co-pulverization technology. By the co-pulverization process, through an appropriate ratio and alternately mixing, the bromhexine hydrochloride soluble powder with good content uniformity and high solubility could be obtained. In addition, results of tests on factors affecting the solubility of the prepared bromhexine hydrochloride, accelerated tests, and long-term tests show that the prepared bromhexine hydrochloride soluble powder has good stability. Further, results of pharmacokinetic and bioequivalence tests of the bromhexine hydrochloride soluble powder in chickens reveal that the prepared bromhexine hydrochloride soluble powder exhibits a linear pharmacokinetic characteristic in chickens, and is bioequivalent with formulations from other countries. This indicates that the bromhexine hydrochloride soluble powder prepared by the method herein has reached an advanced level in China

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 shows a flow chart of a method for preparing a veterinary bromhexine hydrochloride soluble powder according to an embodiment of the present disclosure.

FIG. 2 shows curves of average blood concentration versus time of bromhexine hydrochloride soluble powder (tested) taken orally by chicken for three different doses.

FIG. 3 shows curves of average blood concentration versus time of tested bromhexine hydrochloride soluble powder and a reference formulation which are taken orally by chicken.

### DETAILED DESCRIPTION OF THE INVENTION

In order to make the objects, technical solutions, and advantages of the present disclosure clearer, the present disclosure will be further described in detail below in combination with examples. It should be understood that the specific examples described herein are only used to explain but not to limit the present disclosure.

Aiming at the problems in the prior art, the present disclosure provides a veterinary bromhexine hydrochloride soluble powder, a preparation method and use thereof. The present disclosure will be further described in detail below in combination with drawings.

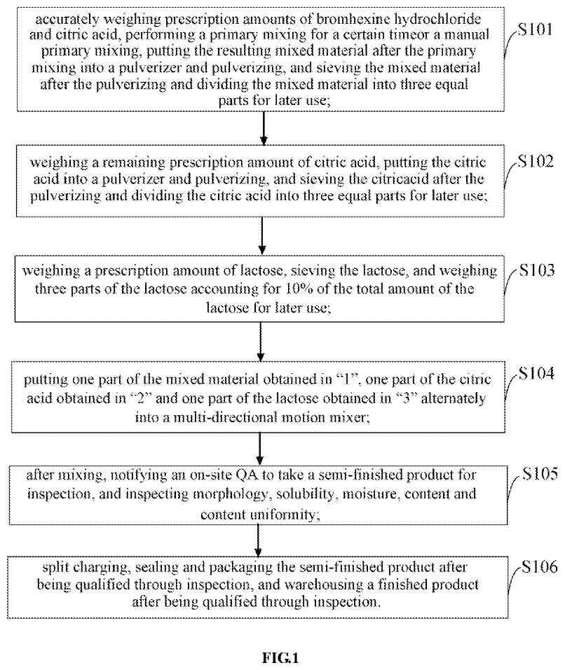

As shown in FIG. 1, a method for preparing a veterinary bromhexine hydrochloride soluble powder according to an embodiment of the present disclosure includes the following steps:

S101: accurately weighing a prescription amount of bromhexine hydrochloride and a part of prescription amount of citric acid, performing a primary mixing for a certain time or a manual primary mixing, putting the resulting mixed material after the primary mixing into a pulverizer and pulverizing, and sieving the mixed material after the pulverizing and dividing the mixed material into three equal parts for later use;

S102: weighing a remaining prescription amount of citric acid, putting the citric acid into a pulverizer and pulverizing, and sieving the citric acid after the pulverizing and dividing the citric acid into three equal parts for later use;

S103: weighing a prescription amount of lactose, sieving the lactose, and weighing three parts of the lactose accounting for 10% of the total amount of the lactose for later use;

S104: putting one part of the mixed material obtained in S101, one part of the citric acid obtained in S102 and one part of the lactose obtained in S103 alternately into a multi-directional motion mixer;

S105: after mixing, notifying an on-site QA to take a semi-finished product for inspection, and inspecting morphology, solubility, moisture, content and content uniformity; and

S106: split charging, sealing and packaging the semi-finished product after being qualified through inspection, and warehousing a finished product after being qualified through inspection.

In S101 of an embodiment of the present disclosure, the citric acid is in an amount of 30% of the prescription amount, and the time for the primary mixing is 10 minutes.

In S101 of an embodiment of the present disclosure, the sieving is performed by a 100-mesh sieve.

In S102 of an embodiment of the present disclosure, the sieving is performed by a 100-mesh sieve.

In S103 of an embodiment of the present disclosure, the sieving is performed by an 80-mesh sieve.

In S104 of an embodiment of the present disclosure, putting the materials into a multi-directional motion mixer is performed as follows: after each one part of the material is added, the materials are mixed for 10 minutes; then the remaining amount of lactose is added at one time, and the materials are mixed for 20 minutes; wherein the content uniformity is monitored depending on the condition of the mixing equipment.

### Example 1

The veterinary bromhexine hydrochloride soluble powder provided by this example consisted of 2 kg of bromhexine hydrochloride, 20 kg of citric acid, and lactose with an amount being supplement to 200 kg.

### Example 2

The veterinary bromhexine hydrochloride soluble powder provided by this example consisted of 2 kg of bromhexine hydrochloride, 22 kg of citric acid, and lactose with an amount being supplement to 200 kg.

### Example 3

The veterinary bromhexine hydrochloride soluble powder provided by this example consisted of 2 kg of bromhexine hydrochloride, 24 kg of citric acid, and lactose with an amount being supplement to 200 kg.

### Example 4

The veterinary bromhexine hydrochloride soluble powder provided by this example consisted of 3 kg of bromhexine hydrochloride, 26 kg of citric acid, and lactose with an amount being supplement to 200 kg.

### Example 5

The veterinary bromhexine hydrochloride soluble powder provided by this example consisted of 3 kg of bromhexine hydrochloride, 28 kg of citric acid, and lactose with an amount being supplement to 200 kg.

### Example 6

The veterinary bromhexine hydrochloride soluble powder provided by this example consisted of 4 kg of bromhexine hydrochloride, 30 kg of citric acid, and lactose with an amount being supplement to 200 kg.

### Example 7

The veterinary bromhexine hydrochloride soluble powder provided by this example consisted of 4 kg of bromhexine hydrochloride, 34 kg of citric acid, and lactose with an amount being supplement to 200 kg.

### Example 8

The veterinary bromhexine hydrochloride soluble powder provided by this example consisted of 4 kg of bromhexine hydrochloride, 40 kg of citric acid, and lactose with an amount being supplement to 200 kg.

The products in the above 8 examples were investigated according to relevant methods of Chinese Veterinary Pharmacopoeia, 2015 Edition for their morphology, solubility, moisture content, related substances, and content. The results are shown in Table 1, which indicate that all the above examples result in formulations with high solubility and good stability.

TABLE 1
Results of quality analysis of different examples

| | Investigated Items | | | | | |
|---|---|---|---|---|---|---|
| Examples | | | | | Total | |
| No. | Morphology | Solubility | Moisture | Impurity I | Impurities | Content |
| 1 | White | Completely | 4.7 | Not | 0.53 | 98.61 |
| | powder | dissolved | | detected | | |
| 2 | White | Completely | 4.8 | Not | 0.52 | 98.75 |
| | powder | dissolved | | detected | | |
| 3 | White | Completely | 4.8 | Not | 0.53 | 99.12 |
| | powder | dissolved | | detected | | |
| 4 | White | Completely | 4.9 | Not | 0.54 | 98.54 |
| | powder | dissolved | | detected | | |
| 5 | White | Completely | 4.7 | Not | 0.55 | 98.48 |
| | powder | dissolved | | detected | | |
| 6 | White | Completely | 4.8 | Not | 0.53 | 99.05 |
| | powder | dissolved | | detected | | |
| 7 | White | Completely | 4.9 | Not | 0.55 | 98.58 |
| | powder | dissolved | | detected | | |
| 8 | White | Completely | 5.1 | Not | 0.56 | 99.21 |
| | powder | dissolved | | detected | | |

In the present disclosure, tests for technical effects and technical solutions were performed. For the product prepared in Example 1, the content uniformity of the prepared bromhexine hydrochloride soluble powder was measured according to the measuring method for content uniformity in Appendix 0941 of Chinese Veterinary Pharmacopoeia, 2015 Edition. The results are shown in Table 2. The results show that the average of content uniformity of the prepared bromhexine hydrochloride soluble powders is 98.65 and relative standard deviation (RSD) is 0.17%, which meets the requirements of Chinese Veterinary Pharmacopoeia, 2015 Edition on content uniformity of soluble powder, indicating that the preparation method could be used in industrialization.

Table 2 Content uniformity of the bromhexine hydrochloride soluble powder

| | Serial Number | | | | | | | | | | | |
|---|---|---|---|---|---|---|---|---|---|---|---|---|
| | 1 | 2 | 3 | 4 | 5 | 6 | 7 | 8 | 9 | 10 | Average | RSD % |
| Content/% | 98.87 | 98.85 | 98.56 | 98.67 | 98.72 | 98.35 | 98.62 | 98.58 | 98.81 | 98.49 | 98.65 | 0.17% |

The product prepared in Example 1 was subjected to strong light irradiation test (illuminance 4500×±5001×), high temperature (40° C.) test, and high humidity (25° C., relative humidity 90%) test respectively, according to “Guiding Principles of Veterinary Drug Stability Test Technology” in Chinese Veterinary Pharmacopoeia, 2015 Edition. The results are shown in Table 3. The results show that the prepared bromhexine hydrochloride soluble powder has no significant changes in content of bromhexine hydrochloride, related substances and moisture after 15 days.

TABLE 3
Results of influence factor test on bromhexine hydrochloride soluble powder

| | | | | | Bromhexine | |
|---|---|---|---|---|---|---|
| | | | | | Hydrochloride | Total |
| Tested Items | | Solubility | Moisture | Content | Impurity I | Impurities |
| Light | 0 days | Meet the | 4.7 | 98.68 | Not detected | 0.53 |
| irradiation | | requirement | | | | |
| test | 5 days | Meet the | 4.8 | 98.72 | Not detected | 0.53 |
| | | requirement | | | | |
| | 10 days | Meet the | 4.7 | 98.65 | Not detected | 0.54 |
| | | requirement | | | | |
| High | 0 days | Meet the | 4.7 | 98.68 | Not detected | 0.53 |
| temperature | | requirement | | | | |
| test | 5 days | Meet the | 4.7 | 98.58 | Not detected | 0.54 |
| | | requirement | | | | |
| | 10 days | Meet the | 4.8 | 98.51 | Not detected | 0.54 |
| | | requirement | | | | |
| High | 0 days | Meet the | 4.7 | 98.68 | Not detected | 0.53 |
| humidity | | requirement | | | | |
| test | 5 days | Meet the | 4.8 | 98.65 | Not detected | 0.54 |
| | | requirement | | | | |
| | 10 days | Meet the | 4.8 | 98.74 | Not detected | 0.53 |
| | | requirement | | | | |

The product prepared in Example 1 was placed at a temperature of 40±2° C. and a relative humidity of 75±5% for 6 months, and samples were taken at 0 day and the end of 1, 2, 3, and 6 months, and subjected to an accelerated test respectively, according to “Guiding Principles of Veterinary Drug Stability Test Technology” in Chinese Veterinary Pharmacopoeia, 2015 Edition. The results are shown in Table 4.

TABLE 4
Results of accelerated test of the bromhexine hydrochloride soluble powder

| Investigated | Time at which the sample was taken and tested | | | | |
|---|---|---|---|---|---|
| Items | 0 day | 1 month | 2 months | 3 months | 6 months |
| Solubility | Meet the | Meet the | Meet the | Meet the | Meet the |
| | requirement | requirement | requirement | requirement | requirement |
| Moisture/% | 4.7 | 4.8 | 4.7 | 4.7 | 4.8 |
| Impurity I/% | Not detected | Not detected | Not detected | Not detected | 0.11 |
| Total | 0.53 | 0.56 | 0.61 | 0.63 | 0.74 |
| Impurities/% | | | | | |
| Content/% | 98.68 | 98.65 | 98.61 | 98.54 | 98.28 |

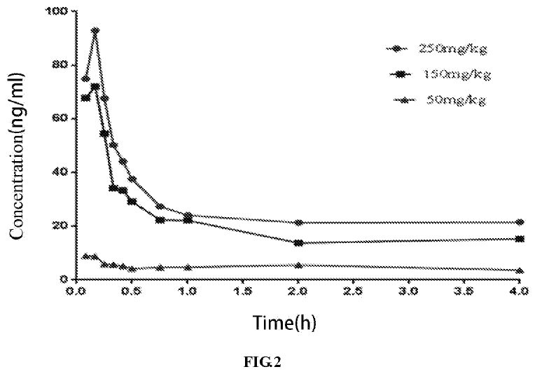

Pharmacokinetic tests of bromhexine hydrochloride soluble powder in chickens were carried out based on three administration dosages of 50 mg/kg·bw, 150 mg/kg·bw and 250 mg/kg·bw. The results show that bromhexine has a linear pharmacokinetic characteristic in chickens. Curves of average blood concentration versus time are shown in FIG. 2.

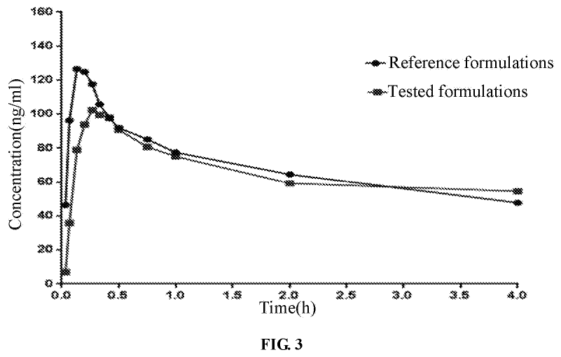

A bioequivalence study was conducted on the products described herein, according to the announcement of Ministry of Agriculture of the People's Republic of China No. 2337 “Quality Management Regulations for Veterinary Drug Clinical Trials” and “Guiding Principles for Bioequivalence Testing of Veterinary Chemicals”, by using 70 healthy chickens which were randomly divided into 2 groups and using QUENTAN POUDRE® produced by Boehringer-Ingelheim as a reference group. Bioequivalence were analyzed using the bioequivalence analysis module of WinNonlin software. The results are shown in FIG. 3 and Table 5.

TABLE 5
Parameters of bioequivalence analysis of the tested bromhexine
hydrochloride soluble powder and reference formulations

90% Confidence interval

| Parameters | P value | CI_90_lower | CI_90_Upper |
|---|---|---|---|
| Ln(AUClast) | 0.52 | 95.02 | 102.14 |
| Ln(AUCobs) | 0.63 | 95.57 | 102.39 |
| Ln(Cₘₐₓ) | 0.48 | 93.18 | 103.51 |

Of note, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, and/or “including,” when used in this specification, specify the presence of stated features, integers, steps, operations, elements, and/or components, but do not preclude the presence or addition of one or more other features, integers, steps, operations, elements, components, and/or groups thereof.

As well, the corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

1. A veterinary bromhexine hydrochloride soluble powder, comprising 1-2% by mass of bromhexine hydrochloride, 8-15% by mass of co-solvent citric acid, with the mass being soluble auxiliary material lactose.

2. A method for preparing the veterinary bromhexine hydrochloride soluble powder, comprising:
step 1: accurately weighing a prescription amount of bromhexine hydrochloride and a part of prescription amount of citric acid, performing a primary mixing for a certain time or a manual primary mixing, putting the resulting mixed material after the primary mixing into a pulverizer and pulverizing, and sieving the mixed material after the pulverizing and dividing the mixed material into three equal parts for later use;
step 2: weighing a remaining prescription amount of citric acid, putting the citric acid into a pulverizer and pulverizing, and sieving the citric acid after the pulverizing and dividing the citric acid into three equal parts for later use;
step 3: weighing a prescription amount of lactose, sieving the lactose, and weighing three parts of the lactose accounting for 10% of the total amount of the lactose for later use;
step 4: putting one part of the mixed material obtained in step 1, one part of the citric acid obtained in step 2, and one part of the lactose obtained in step 3 alternately into a multi-directional motion mixer;
step 5: after mixing, notifying an on-site quality assurer to take a semi-finished product for inspection, and inspecting morphology, solubility, moisture, content and content uniformity; and
step 6: split charging, sealing and packaging the semi-finished product after being qualified through inspection, and warehousing a finished product after being qualified through inspection.

3. The method as claimed in claim 2, wherein in step 1, the citric acid is in an amount of 30% of the prescription amount, and the time for the primary mixing is 10 minutes.

4. The method as claimed in claim 2, wherein in step 1, the sieving is performed by a 100-mesh sieve.

5. The method as claimed in claim 2, wherein in step 2, the remaining prescription amount of citric acid is weighted, put into the pulverizer and pulverized, and the pulverized citric acid is sieved and divided into three equal parts for later use.

6. The method as claimed in claim 2, wherein in step 2, the sieving is performed by a 100-mesh sieve.

7. The method as claimed in claim 2, wherein in step 3, the sieving is performed by an 80-mesh sieve.

8. The method as claimed in claim 2, wherein in step 3, the prescription amount of lactose is weighted, and three parts of lactose accounting for 10% of the total amount of the lactose are weighted.

9. The method as claimed in claim 2, wherein in step 4, putting the materials into a multi-directional motion mixer is performed as follows: after each one part of the material is added, the materials are mixed for 10 minutes; then the remaining amount of lactose is added at one time and the materials are mixed for 20 minutes; wherein the content uniformity is monitored depending on conditions of a mixing equipment.

10. A drug for auxiliary treatment of respiratory diseases of livestock and poultry, wherein the drug is prepared by using a veterinary bromhexine hydrochloride soluble powder comprising 1-2% by mass of bromhexine hydrochloride, 8-15% by mass of co-solvent citric acid, with the mass being soluble auxiliary material lactose.
